(19) United States
(12) Patent Application Publication
(10) Pub. No.: US 2026/0152716 A1
(43) Pub. Date: Jun. 4, 2026

(54) METHODS FOR REDUCTION OF BACTERIAL NUCLEIC ACID CONTENT

(71) Applicant: SUPERBREWED FOOD, INC., New Castle, DE (US)

(72) Inventors: Alon KARPOL, Tel Mond (IL); Shawn JONES, Bear, DE (US)

(21) Appl. No.: 18/706,746

(22) PCT Filed: Nov. 18, 2022
(86) PCT No.: PCT/IB2022/061131

§ 371 Date: May 2, 2024

Related U.S. Application Data
(60) Provisional application No. 63/281,745, filed on Nov. 22, 2021.

(51) Int. Cl. C12N 1/20; C12N 1/36

(52) U.S. Cl. CPC C12N 1/20; C12N 1/36

(57) ABSTRACT

Provided is a method for nucleic acid reduction in a bacterium to less than about 5 wt % of a total dry weight of the bacterium, the method comprising culturing the cells of the at least one bacterium in a fermentation medium to obtain a fermentation medium comprising a biomass; heating the biomass to a temperature of at least 70° C. for at least 10 minutes; and washing the biomass. Also provided are food components, food ingredients and foods or beverages comprising single cell proteins produced from such bacteria.

### CROSS-REFERENCE TO RELATED APPLICATION

The present application gains priority from U.S. Provisional Application No. 63/281,745 filed Nov. 22, 2022 which is incorporated by reference as if fully set-forth herein.

### FIELD OF THE INVENTION

The present invention relates to methods for the reduction of nucleic acid in bacteria and to food products comprising single cell protein (SCP) having such reduced nucleic acid content.

### BACKGROUND OF THE INVENTION

Protein is an essential part of any diet. Increasing world population increases the demand for protein. Vegetable proteins are less attractive sources, since they are relatively poor in some essential amino acids, e.g. lysine, methionine, tryptophan and tyrosine. Animal sources provide the full range of essential amino acids needed by humans in sufficient quantities. The way in which protein is produced has significant impacts on both the environment and human health.

Single-cell protein obtained from microorganisms provides a source of high-value proteins. There is a need for improved methods for the preparation of single-cell protein.

### SUMMARY OF THE INVENTION

According to an aspect of some embodiments of the present invention, there is provided a method for nucleic acid reduction in a bacterium to less than about 5 wt % of a total dry weight of the bacterium, the method comprising:
(i) culturing the cells of the at least one bacterium in a fermentation medium to obtain a fermentation medium comprising a biomass;
(ii) heating the biomass to a temperature of at least 70° C. for at least 10 minutes; and
(iii) washing the biomass.

According to a further aspect of some embodiments of the present invention, there is provided a food component comprising cells of a bacterium, wherein a nucleic acid content of the cells has been reduced according to the method as disclosed herein.

According to a further aspect of some embodiments of the present invention, there is provided a food ingredient comprising the food component as disclosed herein.

According to a further aspect of some embodiments of the present invention, there is provided a food or beverage comprising the food ingredient as disclosed herein.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced.

In the Figures:

FIG. 1 is a bar graph showing reduction of nucleic acid content in C. tyrobutyricum cells following heat treatment at 70° C. for 20 minutes at various pH values.

FIGS. 2A, 2B are bar graphs showing reduction of nucleic acid content in E. callanderi cells following heat treatment for 20 minutes at 70° C. and 90° C., respectively, at various pH values.

### DETAILED DESCRIPTION OF THE INVENTION

The present invention, in at least some embodiments thereof, provides a method for nucleic acid reduction in bacteria.

During production of SCPs by known methods, significant amounts (at least 10% w/w) of nucleic acids (including DNA and RNA) have been found to accumulate within the cells. When such SCPs are used for the preparation of food products, consumption of such levels of nucleic acids may lead to accumulation of uric acid in the body of the consumer, which may potentially lead to gout, kidney stones and related conditions.

Nucleic acid accumulation may also prove problematic during purification of biological drugs for pharmaceutical use.

The present inventors have found that by heating bacterial cells, the nucleic acids contained within are hydrolyzed into monomers, which are small enough to be washed away from the cells, thereby reducing the nucleic acid content of the cells.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise stated, the term “about” as used herein is intended to mean±10%.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a method for nucleic acid reduction in a bacterium to less than about 5 wt % of a total dry weight of the bacterium, the method comprising:
(i) culturing the cells of the at least one bacterium in a fermentation medium to obtain a fermentation medium comprising a biomass;
(ii) heating the biomass to a temperature of at least 70° C. for at least 10 minutes; and
(iii) washing the biomass.

According to some embodiments, the nucleic acid content of the bacterium is reduced to about 4.5 wt %, about 4.0 wt %, about 3.5 wt %, about 3.0 wt %, about 2.5 wt %, about 2.0 wt %, about 1.5 wt %, about 1.0 wt %, about 0.5 wt %, about 0.1 wt %, about 0.05 wt %, about 0.01 wt %, about 0.005 wt %, or even about 0.001 wt %.

According to some embodiments, the biomass is heated to a temperature of at least 70° C., such as about 75° C., about 80° C., about 85° C., about 90° C., about 95° C. or even about 100° C.

According to some embodiments, heating of the biomass is carried out at a pH of at least about 5.6, such as about 5.6, about 5.8, about 6.0, about 6.2, about 6.4, about 6.6, about 6.8, about 7.0 or about 7.2. According to some embodiments, heating of the biomass is carried out at a pH of less than about 7.5

According to some embodiments, the biomass is heated for at least 10 minutes, such as 10 minutes, about 12 minutes, about 15 minutes, about 20 minutes, about 25 minutes, about 30 minutes, about 1 hour, about 1.5 hours, about 2 hours, about 2.5 hours, about 3 hours, about 3.5 hours, about 4 hours, about 4.5 hours, about 5 hours, about 5.5 hours or even about 6 hours.

According to some embodiments, the fermentation medium comprises any suitable fermentation medium known in the art of microbiology for growth of bacteria. Examples of suitable fermentation media include Luria-Bertani (LB) medium, Reinforced Clostridial Medium (RCM), Brain Heart Infusion (BHI) medium, minimal medium and selective medium.

According to some embodiments, the method further comprises separating the biomass from the fermentation medium prior to heating to obtain separated biomass, wherein heating the biomass consists of heating the separated biomass. According to some such embodiments, separating comprises a method selected from the group consisting of microfiltration, centrifugation, agglutination, vacuum separation and combinations thereof. According to some such embodiments, the method further comprises killing at least about 10% of the cells of the separated biomass, such as about 10%, about 15%, about 20%, about 25%, about 30%, about 40%, about 45%, about 50%, about 55%, about 60%, about 65%, about 70%, about 75%, about 80%, about 85%, about 90%, about 95% or about 100% of the separated biomass. According to some embodiments, killing is achieved or performed during heating, such as by pasteurization of the separated biomass. According to some embodiments, killing is performed in addition to heating, either prior to or subsequent to heating.

According to some such embodiments, the method further comprises drying the separated biomass to provide a dry biomass having a water content of no greater than 5 wt %. According to some such embodiments, the dry biomass has a water content of 5 wt %, about 4.5 wt %, about 4.0 wt %,%, about 3.5 wt %, about 3.0 wt %, about 2.5 wt %, about 2.0 wt %, about 1.5 wt %, about 1.0 wt %, about 0.5 wt %, about 0.1 wt %, about 0.05 wt %, about 0.01 wt % or even less than about 0.01 wt %. According to some such embodiments, the method further comprises milling the dry biomass to a particle size of no greater than about 0.5 microns, such as 0.5 microns, about 4.5 microns, about 4.0 microns, about 3.5 microns, about 3.0 microns, about 2.5 microns, about 2.0 microns, about 1.5 microns, about 1.0 microns, about 0.5 microns, or even about 0.2 microns. According to some embodiments, drying is selected from the group consisting of application of hot air (spray drying), indirect or contact (dual drum) drying and combinations thereof.

According to some embodiments, the bacterium is an anaerobic bacterium.

According to some embodiments, the bacterium is a Gram-positive bacterium, optionally of the class Clostridia.

According to some embodiments, the bacterium of the class Clostridia is of an order selected from the group consisting of Eubacteriales and Clostridiales.

According to some embodiments, the bacterium of order Eubacteriales is of the family Clostridiaceae. According to some such embodiments, the bacterium is of the genus Clostridium. According to some such embodiments, the bacterium is of the species Clostridium tyrobutyricum.

According to some embodiments, the bacterium of order Clostridiales is of the family Eubacteriaceae. According to some such embodiments, the bacterium is of the genus Eubacterium. According to some such embodiments, the bacterium is of the species Eubacterium callanderi.

According to an aspect of some embodiments of the present invention, there is provided a food component comprising cells of a bacterium, wherein a nucleic acid content of said cells has been reduced according to the method disclosed herein.

According to some embodiments, there is provided a food ingredient comprising the food component as disclosed herein.

According to some embodiments, there is provided a food or beverage comprising the food ingredient as disclosed herein.

### EXAMPLES

### Example 1: Reduction of Nucleic Acid in a Single Cell Protein Derived from C. tyrobutyricum

Reduction of nucleic acids in cells was tested following heat treatment at different pH levels.

Single cell proteins derived from Clostridium tyrobutyricum were incubated at 70° C. for 20 minutes, at pH 5.80, 6.75, 7.00 or 7.25, then cooled in ice water, after which the nucleic acid content was measured. For comparison, for each pH value, the nucleic acid content after heat treatment was compared to that of cells maintained at 4° C. for 20 minutes (non-heat treated group) at each of the sane pH levels as those used for the beat treated groups. Nucleic acid measurement was carried out as follows.

Samples comprising 500 g of wet material (approximately 10% solids) or 50 mg of dried material were each introduced into a 15 ml Falcon tube. 2.5 ml of 0.6M HClO₄ was added. 500 μl of 1 mM Allopurinol (Sigma Chemical, St. Louis, MO, USA) was used as the internal standard. Samples were vortexed for 10 seconds.

The tube was incubated in a water bath at 90-95° C. for 1 hour, then removed from that water bath and cooled to room temperature.

1 ml of sample was spun down and filtered under sterile conditions. The filtered samples was loaded onto a HLPC column and separation performed at 40° C.

3 ml of mobile phase A was added. The pH was measured and adjusted to a value in the range of 6.6-6.9 with 8M KOH.

The mobile phases used were: (a) 10 mm NH4H2PO4 adjusted to pH 6.0 using 2.86 M NH4OH, and (b) methanol in a gradient elution from 2% to 10% of (b) during 20 min (1 ml/min), followed by a 5-minute equilibration using the starting conditions.

HPLC setup phases were as follows:

$$Flow Rate = 1 mL / min$$

Mobile Phase A: 10 mM NH4H2PO4 pH=6.0 (98%)
Mobile Phase B: 100% methanol
Storage Solution: 34:66 water:methanol
   Data were analyzed against external standards (adenine, guanine, cytosine, thymine, uracil and allopurinol) according to Table 1.

TABLE 1

| Adenine | Cytosine | Guanine | Thymine | Uracil | Allopurinol |
|---|---|---|---|---|---|
| Conc | Conc | Conc | Conc | Conc | Conc |
| (mM) | (mM) | (mM) | (mM) | (mM) | (mM) |
| 1 | 1 | 1 | 1 | 1 | 1 |
| 0.5 | 0.5 | 0.5 | 0.5 | 0.5 | 0.5 |
| 0.2 | 0.2 | 0.2 | 0.2 | 0.2 | 0.2 |
| 0.1 | 0.1 | 0.1 | 0.1 | 0.1 | 0.1 |
| 0.05 | 0.05 | 0.05 | 0.05 | 0.05 | 0.05 |
| 0.01 | 0.01 | 0.01 | 0.01 | 0.01 | 0.01 |

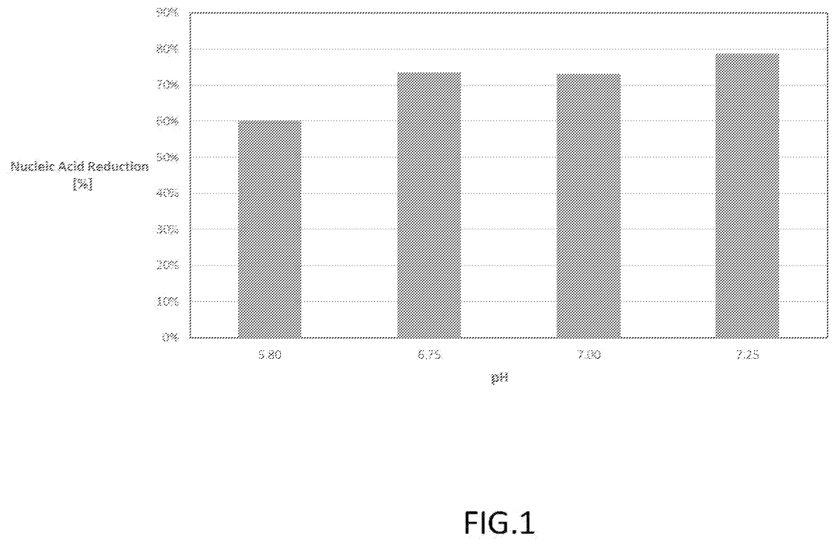

Results are presented in FIG. 1.

As shown in FIG. 1 and Table 2, the non-heat treated group showed no reduction in nucleic acid content, while a reduction of at least 60% of the initial nucleic acid content was seen in the heat treated groups at each pH value.

TABLE 2

| | | |
|---|---|---|
| pH | Temperature | Nucleic acid content (%) |

| | | | |
|---|---|---|---|
| 5.8 | 4° | C. | 8.87 |
| | 70° | C. | 3.53 |
| 6.75 | 4° | C. | 8.97 |
| | 70° | C. | 2.39 |
| 7.0 | 4° | C. | 9.26 |
| | 70° | C. | 2.49 |
| 7.25 | 4° | C. | 8.86 |
| | 70° | C. | 1.88 |

### Example 2: Reduction of Nucleic Acid in a Single Cell Protein Derived from E. callanderi

Single cell proteins derived from Eubacterium callanderi were treated at pH of 5.6 and 7, and temperature of 70° C. or 90° C., and nucleic acid content measured as described in Example 1 for Clostridium tyrobutyricum.

For comparison, for each pH value, the nucleic acid content after heat treatment was compared to that of cells maintained at 4° C. for 20 minutes (non-heat treated group) at each of the same pH levels as those used for the heat treated groups. Nucleic acid measurement was carried out as described in Example 1 above.

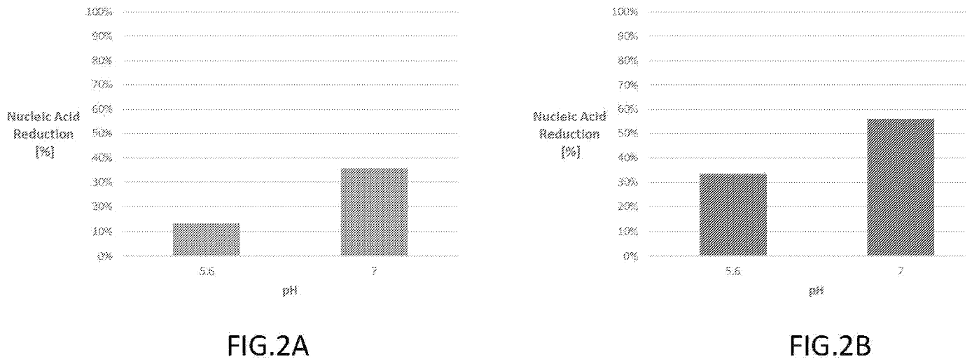

Results are presented in FIG. 2 and Table 3.

As shown in FIG. 2, a statistically significant reduction in nucleic acid content was obtained at a temperature of at least 70° C. at each of the pH values tested, with a greater reduction at higher pH values and higher temperatures.

TABLE 3

| | | |
|---|---|---|
| pH | Temperature | Nucleic acid content (%) |

| | | | |
|---|---|---|---|
| 5.6 | 4° | C. | 6.65% |
| | 70° | C. | 5.69% |
| | 90° | C. | 4.36% |
| 7.0 | 4° | C. | 6.63% |
| | 70° | C. | 4.22% |
| | 90° | C. | 2.9% |

As shown in FIG. 2 and Table 3, at pH 5.6 a reduction in nucleic acid content of 15% and 34% was obtained at temperatures of 70° C. and 90° C., respectively, as compared to the non-heat treated group; while at pH 7.0, a reduction in nucleic acid of 36% and 56% was obtained at 70° C. and 90° C., respectively, as compared to the non-heat treated group.

1. A method for nucleic acid reduction in a bacterium to less than about 5 wt % of a total dry weight of said bacterium, the method comprising:
(i) culturing said cells of at least one bacterium in a fermentation medium to obtain a fermentation medium comprising a biomass;
(ii) heating said biomass to a temperature of at least 70° C. for at least 10 minutes; and
(iii) washing said biomass.

2. The method of claim 1, further comprising separating said biomass from said fermentation medium prior to said heating to obtain separated biomass, wherein said heating said biomass consists of heating said separated biomass.

3. The method of claim 2, wherein said separating comprises a method selected from the group consisting of microfiltration, centrifugation, agglutination, vacuum separation and combinations thereof.

4. The method of claim 3, further comprising killing at least 50% of said cells of said separated biomass.

5. The method of claim 4, wherein said killing is achieved during said heating.

6. The method of claim 4, wherein said killing is performed in addition to said heating.

7. The method of claim 6, wherein said killing is performed prior to said heating.

8. The method of claim 6, wherein said killing is performed subsequent to said heating.

9. The method of claim 5, wherein said killing comprises pasteurizing said separated biomass.

10. The method of claim 2, further comprising drying said separated biomass to provide a dry biomass having a water content of no greater than 5 wt %.

11. The method of claim 10, further comprising milling said dry biomass to a particle size of no greater than about 0.5 microns.

12. The method of claim 1, wherein said at least one bacterium is an anaerobic bacterium.

13. The method of claim 1, wherein said at least one bacterium is a Gram-positive bacterium.

14. The method of claim 1, wherein said Gram-positive bacterium is of the class Clostridia.

15. The method of claim 1, wherein said bacterium is a species selected from the group consisting of Clostridium tyrobutyricum and Eubacterium callanderi.

16. A food component comprising cells of a bacterium, wherein a nucleic acid content of said cells has been reduced according to the method of claim 1.

17. A food ingredient comprising the food component of claim 16.

18. A food or beverage comprising the food component of claim 17.
